(19) United States
(12) Patent Application Publication
(10) Pub. No.: US 2021/0379557 A1
(43) Pub. Date: Dec. 9, 2021

(54) A SYSTEM AND METHOD FOR PROMOTING CHEMICAL REACTIONS

(71) Applicants: Yeda Research and Development Co. Ltd., Rehovot (IL); Yissum Research Development Company of the Hebrew University of Jerusalem Ltd., Jerusalem (IL)

(72) Inventors: Ron NAAMAN, Yarkona (IL); Claudio FONTANESI, Rehovot (IL); Francesco TASSINARI, Rehovot (IL); Suryakant MISHRA, Rehovot (IL); Eyal CAPUA, Rehovot (IL); Yossef PALTIEL, Maskeret Batya (IL); Shira YOCHELIS, Ness Ziona (IL); Tzuriel METZGER, Jerusalem (IL)

(21) Appl. No.: 17/288,460

(22) PCT Filed: Oct. 23, 2019
(86) PCT No.: PCT/IL2019/051143

§ 371 Date: Apr. 23, 2021

Related U.S. Application Data
(60) Provisional application No. 62/749,271, filed on Oct. 23, 2018.

(51) Int. Cl. B01J 19/08; B01J 19/22; C07C 319/28; C07C 319/02

(52) U.S. Cl. CPC B01J 19/087; B01J 19/22; C07C 319/28; B01J 2219/0877; B01J 2219/0809; B01J 2219/0852; B01J 2219/0875; C07C 319/02

(57) ABSTRACT

A system and method for use in synthesis and promoting interactions of chiral molecules. The system can include: a container configured for containing fluid mixture comprising one or more reactant molecules and at least one surface comprising ferromagnetic or paramagnetic material located to be in at least partial contact with reactants in said container. The ferromagnetic of paramagnetic material can be magnetizable with magnetization direction perpendicular to said at least one surface, thereby providing chiral selective synthesis from said one or more reactant molecules. The technique can enable selective interactions of enantiomers of selected handedness of chiral molecules or formation of selected enantiomers from achiral molecule reactants.

### CROSS REFERENCE TO RELATED APPLICATION(S)

This application is a national phase filing under 35 C.F.R. § 371 of and claims priority to PCT Patent Application No. PCT/IL2019/051143, filed on Oct. 23, 2019, which claims the priority benefit under 35 U.S.C. § 119 of U.S. Provisional Application No. 62/749,271 filed on Oct. 23, 2018, the contents of each of which are hereby incorporated by reference in their entireties.

### BACKGROUND

Some embodiments are directed to the field of systems and methods for promoting selected chemical interactions and relates in particular to techniques for promoting enantio-specific reactions of chiral molecules having selected handedness thereby allowing separation of chiral molecules and selection of molecules of desired handedness.

### BACKGROUND

Chiral molecules are molecules having non-supposable mirror reflection, generally allowing two non-identical enantiomers which are mirror reflections of each other. Biological systems are typically based on molecules with specific chiral properties. More specifically, in many cases, enantiomers of one handedness interact in biological systems with specific receptors, while the other handedness may not interact, or interact with receptors to provide undesired outcome.

General chemical processes, performed outside of biological systems, are often non-selective to chirality. Thus, reactants in a chemical reaction may include all or most enantiomers of chiral molecules, or the reaction forms both enantiomers of molecules without selectivity to handedness of the products.

Generally, promoting enantio-specific chemical processes, may require either enantio-pure reagent or an environment that has a specific handedness (mainly chiral environment with a specific chirality). Accordingly, the conventional techniques for producing chiral molecules of selected handedness rely on either applying chiral specific catalyst or reaction with an enantio-pure reagent.

It was recently found that when chiral molecules are electrically polarized by electric field, the electric polarization is accompanied by spin polarization. Forming a state where at each electric pole there is an unpaired electron, or part of an electron, which for some finite time has a specific spin orientation that depends on the specific chirality of the molecule. In addition, it was found that when chiral molecules are adsorbed on a ferromagnetic substrate, which is initially not magnetized, the substrate tends to magnetize, and the direction of the magnetic dipole depends on the specific chirality. Namely, one enantiomer will cause the substrate to be magnetized with the magnetic dipole pointing up, whereas the other enantiomer will cause the dipole to point down.

### SUMMARY

There is a need in the art for a novel technique for use in chemical and molecular synthesis, allowing chirality selective synthesis. Generally, the present technique enables chemical synthesis using chiral reactants of specific handedness (specific enantiomer) and/or achiral molecules with a chiral catalyst.

Spin polarized electrons can be characterized by helicity property. The helicity of a particle relates to relations between the angular momentum (spin) and linear momentum. Moving spin polarized electrons have well defined helicity that depends on the sign of the spin. The helicity of electrons has similar symmetry as that of a chiral molecule with corresponding handedness. This effect indicates that based on symmetry considerations the spin polarized electron can provide “chiral environment” that can promote interaction or production of one enantiomer versus the other. More specifically, the spin polarized electrons are used as chiral reagents for enantiospecific reactions. This omits the need for using enantio-pure chiral catalysts.

To this end, the present technique provides an interaction container configured for containing one or more reactant molecules and including at least one magnetized surface having magnetization in direction perpendicular to the surface. Location and shape of the surface with respect to regions where the interaction takes place, as well as chemical or electrical properties thereof are selected to bring the reactant molecules to interact at the vicinity of the surface. The magnetization of the at least one magnetized surface affects spin distribution of the electrons participating in the chemical interaction, resulting in interaction preference toward selected handedness of chiral reactants.

To this end, in some embodiments of the technique, the at least one magnetized surface is configured as electrically conducting surface connected to an electrical power supply unit, and the container includes at least one additional electrode providing opposite electrical potential. In this configuration, electrochemical interactions of reactant molecules involve donating or receiving electrons from the at least one magnetized surface. Due to perpendicular magnetization of the surface, such interaction is specific to electrons having spins that are in-line with magnetization of the surface, providing selectivity to chiral reactants of corresponding handedness.

In some other configurations, selected reactants are pre-adsorbed onto the at least one magnetizes surface. Magnetization of the at least one magnetized surface induces spin polarization on electrons at interacting ends of the adsorbed reactants, thereby promoting interaction forming chiral products of specific chiral handedness. Generally, to maintain spin polarization at the interacting ends of the adsorbed reactants, location of the interacting ends of the adsorbed reactant is desired to be no more than several angstroms away from the magnetized surface.

Generally, the electron spin providing the fermionic nature of electrons, is key to the Pauli Exclusion principle and hence the stability of matter. Because of its spin, the electron has a magnetic moment, and a magnetic field is usually required to separate (or split) the energies of its two possible spin states (quantum numbers of ½ or −½). However, this splitting energy is typically small compared to the ambient thermal energies (about 0.025 eV). In contrast, the interaction energy between two electrons occupying the same volume can be large, on the order of an eV (about 20 kJ/mole) and it arises from the exchange/correlation energy, not the magnetic interactions. A manifestation of this energy is the energy gap between singlet and triplet states, in which the spins are antiparallel for the singlet and parallel for the triplet.

The electron's spin is considered as an one of the important venues for transferring information, replacing the electron's charge. In addition, the spin serves in memory applications and is a candidate as one of the important tools to introduce quantum mechanics into computation. Any attempt to utilize spin dynamics may require material through which the spin information can be transferred. In most cases, the spin transport was attempted by transferring electron that carries a pre-defined spin through a conductor or semiconducting materials. In another approach, no charge is actually transferred but the spin information is transferred by exchange interaction through the system. Organic molecules are not generally considered as a good medium for transferring neither charge nor spin information. However, the present technique utilizes spin polarization and transfer properties of electrons in organic molecules, being either achiral or chiral molecules, to utilize relation between spin polarization and chirality for promoting reaction of the molecules with, or near, one or more magnetic surfaces. For example, the technique may utilize interaction between chiral or achiral molecules adsorbed on the surface and one or more types of chiral molecule in a racemic mixture in contact with the adsorbed molecules.

Because of the significant exchange energy differences associated with the relative spin alignment of electrons, the interaction energy between the electrons might be considered to be useful for controlling chemical reactions. However, it has been demonstrated that except for photochemical and some enzymatic reactions, the interaction energy between the electrons cannot be used for controlling chemical reactions. The reason is that the direction of a spin is typically not defined in the molecular frame and is well defined with respect to external magnetic field. When the magnetic field's direction changes, the same electron's spin may be defined again irrespective of its direction relative to previous magnetic field. Thus, the spin direction is generally not defined in the molecular frame. The exception is the case when unpaired electrons reside on the same molecule. Then, the relative spin directions are defined in the context of the unpaired electrons giving rise to a singlet, a triplet, or a higher multiplet states.

This difficulty in relating spin orientation to the molecular frame generally limits the ability to exploit spin polarization for controlling reactions between two molecules or between molecules and a surface. The recently discovered chiral induced spin selectivity (CISS) effect implies that chiral molecules (or moieties) have a preferred orientation of the electron spin in the molecular frame, having significant implications for controlling chemical processes, enantiomer separations, and bio-recognition processes.

When a chiral molecule interacts with another molecule or with a surface, the charge reorganization associated with this reorganization results in a transient spin polarization in the molecule, because the electronic redistribution in the molecule is spin dependent. In this connection, it should be understood that when a molecule approaches a surface, the molecule is charge polarized. For chiral molecules, this charge polarization is accompanied by transient spin polarization. The spin direction associated with each electric pole depends on the handedness of the molecule. If the substrate is a ferromagnet, magnetized perpendicular to its surface, only one enantiomer is able to bind to the surface or to accept electron from the surface. Hence the interaction with the surface is enantio-selective. This effect results in the ability to separate enantiomers from racemic mixture, using ferromagnetic surfaces as described for example in K. Banerjee-Ghosh et al “Separation of enantiomers by their enantiospecific interaction with achiral magnetic substrates”, Science 360, 1331-1334 (2018) and in WO 2019/043,693 describing aspects use of a relation between spin polarization and adsorption selectivity. The racemic mixture can thus be separated to its two different enantiomers by adsorbing preferentially one enantiomer on the substrate magnetized perpendicular to its surface.

For having enantio-specific chemical process, at least one pure enantiomer of a chiral molecule, or enantio-pure chiral catalyst need to be involved. It was shown that photoelectrons transmitted through chiral monolayer of double stranded DNA, induce manner dissociation of chiral molecules in enantio-selective. As described above, it has been shown that enantio-selective adsorption on an achiral substrate can be controlled by the substrate being magnetic with magnetization perpendicular to its surface. The presently disclosed subject matter utilizes the CISS effects for inducing enantio-specific reactions (e.g. redox reactions) in a racemic mixture of chiral molecules in an electrochemical cell.

It is known that electrode coated with chiral molecules or films may show enantio-specific interaction with the redox couple in solution. This is viewed as a result from the common chiral-recognition properties that result from spatial preferred interaction between the chiral molecules on the electrode and the chiral redox couple. It is therefore expected, that for each specific chiral molecule, a specific chiral coating on the electrode will result in enantio-selectivity.

The presently disclosed subject matter provides a system and method enabling carrying out a chemical reaction while promoting reactions of enantiomers of certain handedness over enantiomers of the opposite handedness. More specifically, in some configurations, the reactants include at least one chiral molecule and the technique promotes the reaction with one enantiomer of the molecule over the other. In some other configurations, the products include achiral molecules and the technique promotes reaction toward selected handedness of the product.

The technique is based on providing reactants in a container having at least one magnetic surface (surface including ferromagnetic or paramagnetic material) having magnetization in a direction perpendicular to the surface. The container is configured to allow the chemical reaction to occur at a distance that does not exceed several angstroms from the surface, effectively allowing transmission of electrons with one spin orientation (with respect to the direction of magnetization). This may be used in electrochemical reactions where the spin polarized electrons can promote reaction selectively with one enantiomer or produce chiral product from achiral reactant or in a case that no current is flowing, and achiral or chiral molecules are adsorbed on a substrate magnetized perpendicular to the surface and the adsorbed molecules react spontaneously in enantio-specific manner with chiral molecules in the solution.

Moreover, the inventors have demonstrated that organic molecules can serve for transferring spin information with high reliability and the transferred information can be utilized to control the interaction of the molecule with chiral molecules. Hence, the system of the presently disclosed subject matter provides a mean for controlling chemical interaction, using the electron's spin. In this connection, it should be understood that magnetic proximity effects can transform a given material to become magnetic through its adjustment to a magnet. In bulk materials, the sample size exceeds significantly the characteristic lengths of proximity effects, allowing their neglect. However, in the case of monolayers even short-range magnetic proximity effects may have a range comparable with their thickness. The inventors have shown that in the case of self-assembled organic monolayers, the magnetic proximity effect may induce selectivity in their reactions with chiral molecules. The selectivity means that for magnetic field pointing perpendicular to the surface and away from it, the reaction will occur with one enantiomer while if the magnetic field is pointing to the opposite direction, the other enantiomer will react. The selectivity depends on the thickness of the organic monolayer. Interestingly, the extend of control on the achiral-chiral interaction does not depend on the nature of the bond formed and it is shown up when the bonds are covalent, π-π interaction, or hydrogen bond.

Thus, according to one broad aspect, the presently disclosed subject matter provides a system for use in synthesis of molecules, including: a container configured for containing fluid mixture including one or more reactant molecules, and at least one surface including ferromagnetic or paramagnetic material, located to be in at least partial contact with reactants in said container, said ferromagnetic or paramagnetic material being magnetized with magnetization direction perpendicular to said at least one surface, thereby providing chiral selective synthesis from said one or more reactant molecules.

Generally, the at least one surface may include structured substrate including at least one layer of ferromagnetic or paramagnetic material. The structured substrate may further include at least one surface layer having affinity for adsorption with one or more selected reactants, thereby enabling molecular interactions between reactants at vicinity of the at least one surface.

According to some embodiments, the system may further include at least one type of reactant molecules pre-adsorbed on said at least one surface, said pre-adsorbed reactant molecules selectively interacting with molecules of selected handedness in said fluid mixture. The at least one type of reactant molecules pre-adsorbed on said at least one surface includes molecules having length that does not exceed certain length (e.g. corresponding to a few carbon link length).

According to some embodiments, the system may further include one or more additional types of reactant molecules being adsorbed in corresponding one or more additional layers associated with enantiospecific interactions. In some cases, each of the additional layers adds certain thickness, while maintaining spin polarization properties.

According to some embodiments, the at least one surface may be associated with an electrode providing electrical contact with said fluid mixture, thereby promoting electro-chemical reaction at the vicinity of the at least one surface.

According to some embodiments, the at least one surface including ferromagnetic or paramagnetic material is mounted to be selectively placed to be in contact with reactants in said container, and to be placed out of contact with said reactants.

According to some embodiments, the system may be configured for selectively promoting chemical reactions for enantiomers of selected handedness of chiral molecules in accordance with direction of magnetization perpendicular to said at least one surface being up or down with respect to said at least one surface.

According to one other broad aspect, the presently disclosed subject matter provides a system for use in synthesis of molecules, the system including:
(a) a container configured for containing fluid mixture that including one or more types of chiral molecules;
(b) an electrode arrangement including at least first and second electrodes, being configured for applying an electric field on said fluid mixture; said first electrode includes at least one ferromagnetic or paramagnetic material providing at least one interface with said fluid mixture, said at least one ferromagnetic or paramagnetic material being magnetized (e.g. selectively magnetized) in up or down direction perpendicular to surface of said first electrode; said second electrode being located at a predetermined distance from said first electrode and being electrically insulated therefrom; wherein magnetization of said first electrode provides spin selectivity in electrons transmission in interaction with molecules in said fluid mixture, thereby providing enantioselective interaction with molecules in said fluid mixture;
(c) a power unit configured for providing electrical voltage and current between said first and second electrodes.

According to some embodiments, the first electrode may include at least one ferromagnetic or paramagnetic material, said system further includes at least one field generating unit configured to selectively apply magnetic field onto said first electrode providing selected magnetization direction of said first electrode.

Generally, the system may be configured for synthesis or separation of chiral molecules. For example, the system may be configured for enantiospecific reactions such of selected enantiomer from racemic mixture. Such enantiospecific reaction may be electrochemical oxidation or reduction reactions.

According to some embodiments, the at least said first electrode is mounted to be selectively insert into said container for selectively start and stop said interaction with reactants in said fluid mixture.

According to yet another broad aspect, the presently disclosed subject matter provides a method for use in molecular synthesis, the method including: providing at least one substrate having magnetization perpendicular to surface of said substrate; bringing said substrate in contact with one or more types of reactant molecules for promoting one or more molecular reactions on or in vicinity of surface of said substrate; wherein magnetization direction of said at least one substrate provides spin selectivity of electrons participating in said one or more molecular reactions resulting in enantioselective reactions.

According to some embodiments, said one or more molecular reactions may include electro-chemical reaction, the method further includes applying selected electrical voltage on said at least one substrate thereby receiving or donating electrons of selected spin polarization to perform said one or more molecular reactions.

According to some embodiments, the method may further include adsorbing one or more selected reactants onto said surface of said at least one substrate prior to bringing said substrate in contact with one or more types of reactant molecules, thereby providing enantioselective interactions between said one or more types of reactant molecules and said one or more selected reactants adsorbed on said surface.

According to some embodiments, said one or more types of reactant molecules may include chiral molecules, said enantioselective reactions provide reactions of enantiomers of one handedness selected in accordance with direction of magnetization of said at least one substrate.

According to some embodiments, said one or more types of reactant molecules may include a-chiral molecules, said enantioselective reactions provide formation of chiral products having handedness selected in accordance with direction of magnetization of said at least one substrate.

According to some embodiments, the method may be configured for enantiomer separation by physical adsorption enantiomers of selected handedness.

Generally, according to some embodiments, the method may include transferring said least one substrate to container including buffer solution for removing molecules of said selected handedness from said least one substrate.

According to yet another broad aspect, the presently disclosed subject matter provides a method for use in separation of chiral molecules, the method including: providing at least one substrate having magnetization perpendicular to surface of said substrate; bringing said substrate in contact with mixture of chiral molecules for a selected time and removing said substrate from said mixture to remove the adsorbed molecules therefrom; wherein magnetization direction of said at least one substrate provides preferred adsorption of molecules of one enantiomer over molecules of the opposite enantiomer thereby removing molecules of a selected enantiomer from said mixture.

According to some embodiments, the method may further include, washing adsorbed molecules from said substrate in a second container, thereby providing a solution of substantially enantiomer pure mixture.

According to some embodiments, the method may further include, repeatedly bring said substrate into contact with said mixture of chiral molecules for selected time and washing said substrate in a second container to thereby provide a continuous separation process.

According to some embodiments, the method may further include, using a plurality of substrates and repeating said separation process with each of said substrates.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A and 1B show experimental results indicating enantioselective reactions of chiral molecules from racemic mixture, FIG. 1A shows absorption variation before and after reaction, and FIG. 1B shows Circular Dichroism (CD) spectra indicating enantioselective reaction;

FIG. 2 exemplifies relation between electron helicity and structure of chiral (e.g. helical) molecule;

FIG. 3 illustrates schematically a system for enantioselective molecular interaction according to some embodiments of the presently disclosed subject matter;

FIGS. 4A to 4B show experimental results of enantioselective molecular interaction according to some embodiments of the presently disclosed subject matter, FIG. 4A illustrates the experimental system, and FIG. 4B exemplifies Fourier Transform Infrared spectroscopy (FTIR) results indicating enantioselective interaction;

FIGS. 5A to 5F exemplify additional results of Fourier Transform Infrared spectroscopy (FTIR) for selected reaction time and magnetic direction properties; and

FIGS. 6A to 6C illustrate additional example of device configuration including a layered Hall device (FIG. 6A) and Hall voltage (FIG. 6B) and mobility (FIG. 6C) measurements thereof further indicating enantioselective interaction provided according to some embodiments of the presently disclosed subject matter.

### DETAILED DESCRIPTION OF EMBODIMENTS

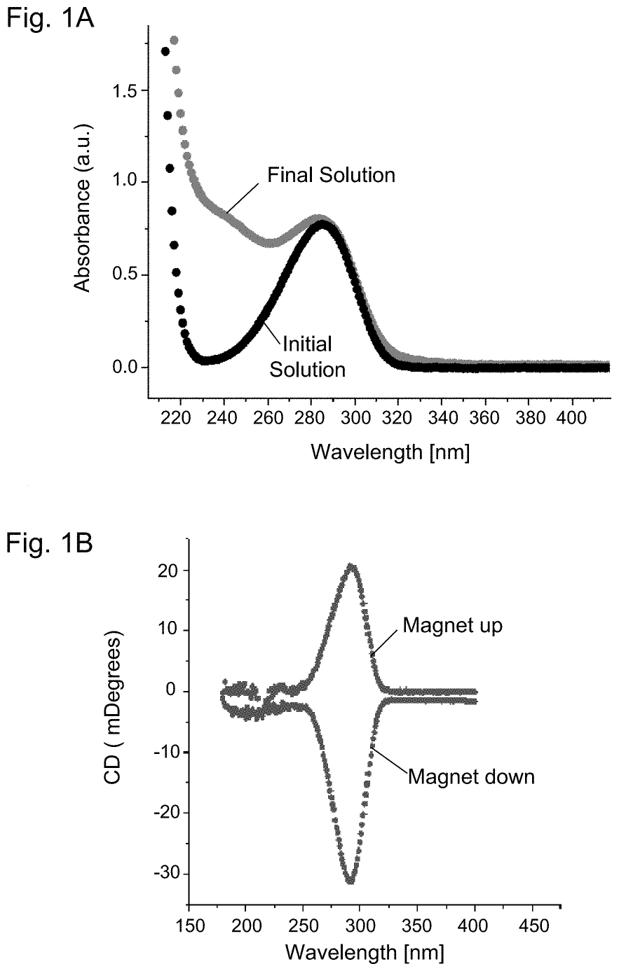

Reference is made to FIGS. 1A-1B illustrating a Circular Dichroism (CD) spectra recorded after electrochemical reduction of racemic camphor sulfonic acid to a borneol form using a nickel electrode magnetized with its normal pole either up or down with respect to the normal to the electrode surface. The reduction reaction was carried out in a 20 mM racemate solution at a constant potential of −0.9 V vs Ag/AgCl for 360 minutes. FIG. 1A shows a UV-Vis absorption spectroscopy of the racemic camphor sulfonic acid before (Initial Solution) and after the reaction (Final Solution). FIG. 1B shows the CD spectra obtained after the reaction for products obtained when the magnetization of the nickel electrode points with its north pole up (Magnet up) or down (Magnet down). The absorption spectrum changed, as shown in FIG. 1A indicating the existence of reaction product in the sample. The initial racemic mixture shows no circular dichroism (CD) signal. Accordingly, after being exposed to spin polarized current for a certain time, a CD signal appears and is correlated with the direction of magnetization of the electrode. When the magnetic electrode is magnetized with its north pole in up direction, perpendicular to surface of the nickel electrode the CD signal is positive, while when the electrode is magnetized with the north pole down, the CD signal is negative.

The obtained CD signals indicate that after the electrochemical reaction, one enantiomer remains in the solution, while the molecules of the other enantiomer have been reduced, providing increase CD signal with respect to the racemic mixture. This result demonstrates enantioselective electrochemical redox process of camphor sulfonic acid, so that one enantiomer was reduced and the other remain mainly unaffected. It should be noted that the enantioselectivity in electrochemical reaction (e.g. electro-oxidization, electro-hydrogenation) is not specific to camphor sulfonic acid and may be used with various other molecules.

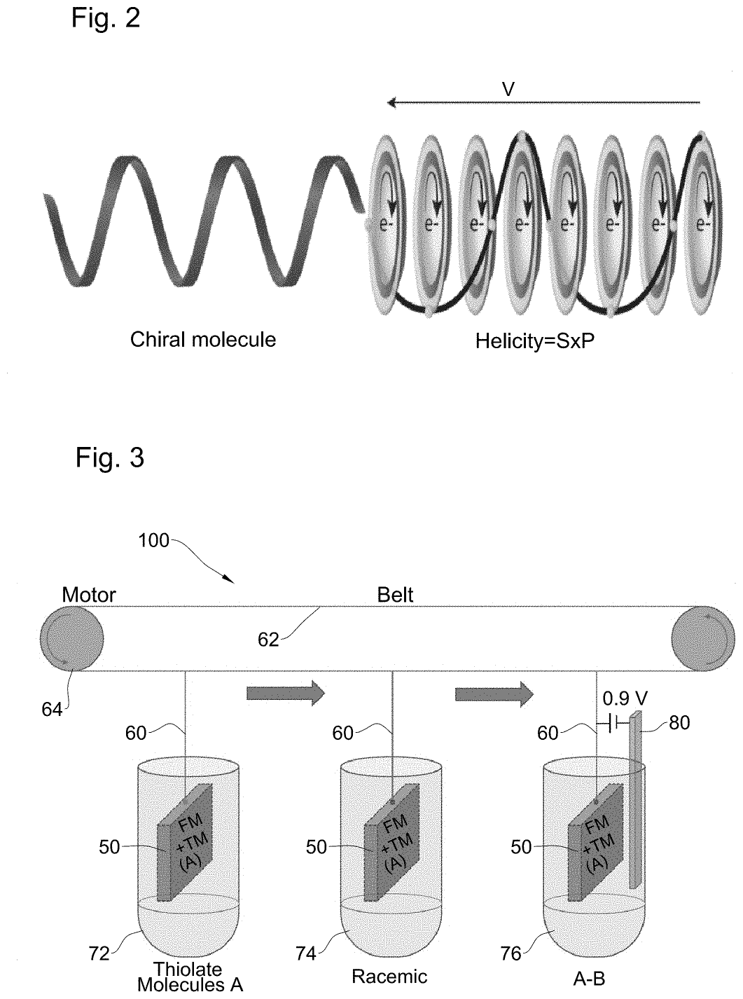

As described above, when a molecule approaches a substrate (being a surface or other molecules), charge rearrangement occurs, and an induced dipole is formed, in which fractions of an electron or hole is located at the negative and positive poles respectively. A moving electron with its spin has a certain helicity, i.e. relation between the spin and the linear momentum of the electron. The similarity between the electron helicity and chirality of a molecules is exemplified in FIG. 2 illustrating chiral (helical) molecule and electron helicity. As can be seen in the figure, helicity of the electron and that of the molecules may be of same or opposite handedness, affecting transmission properties of the electron through the molecules. Thus, the spin polarized electrons may act as chiral reagents for enantiospecific interactions omitting the need for enantio-pure chiral catalyst.

Typically, if the ground state of the electron is a singlet state, the spins at the electric poles will be anticorrelated, so that the total spin of the molecule remains zero. As described above, this effect varies with the adsorption of molecules on the magnetized substrates depending on the spin of the electron pointing towards the surface. Accordingly, for a chemical bond to be formed between the molecule and the substrate, the electron that is donated by the molecule and the one donated by the magnetized substrate to the bond formation, should be of opposite spins. This effect is similar upon interaction between two molecules. The electrons in the magnetized substrate that can participate in the bond with the molecule, have a well-defined spin due to magnetization of the substrate. Therefore, the electron donated by the molecule must or should have the opposite spin. This leaves the molecule with (partially) unpaired electron in the electric pole farther away from the substrate and this electron has the same spin as the ferromagnetic layer, as long as the system is coherent.

The present technique utilizes such spin preference in interaction between molecules and magnetized substrates (or between molecules in the vicinity of magnetized substrates). Such spin preference holds not only for covalent bonding but for any strength of interaction between the molecules and the ferromagnetic substrate, being adsorption, electron exchange, etc. More specifically, magnetized surface/substrate can donate or receive electron of a selected spin. Additionally, when molecules are adsorbed on the surface, the unpaired electron located on the opposite pole of the molecule, away from the surface, is generally similarly spin polarized. This extends the effect of the surface magnetization and resulting chiral selectivity to molecule-molecule interaction. More specifically, such unpaired, spin polarized electron of molecules may interact in enantio-specific manner with additional chiral molecules, as well as with non-chiral molecules. This enables promoting chiral selective interactions, where chiral molecules of selected handedness participate in the interaction, while same molecules with opposite handedness do not take part in the reaction. Additionally, such spin selectivity of the electrons facilitating chemical reaction, may lead to selected chiral symmetry of reaction products, even if the initial reactants are non-chiral.

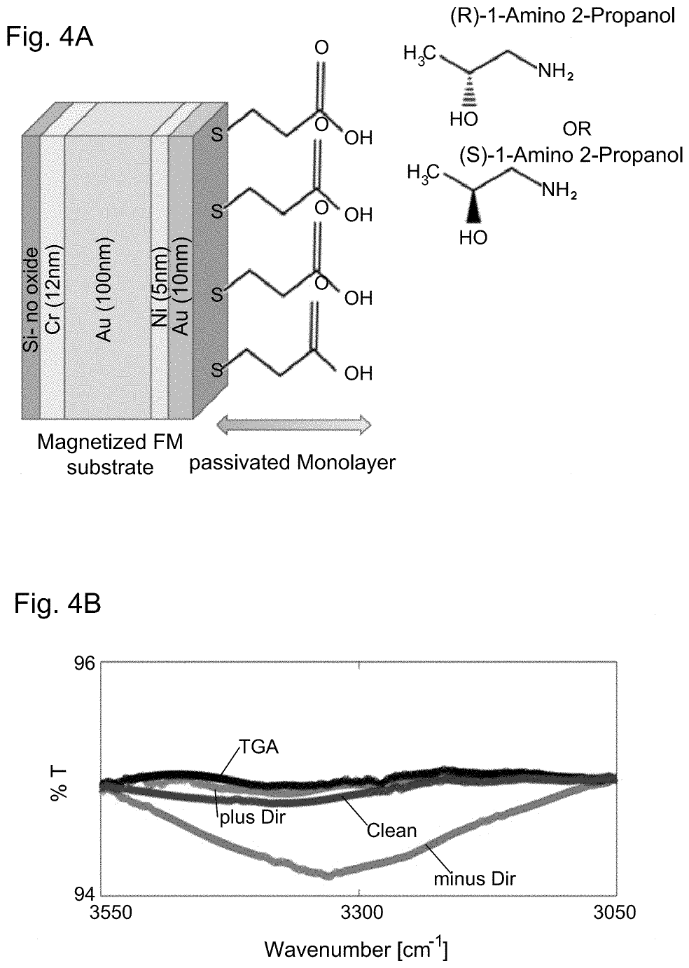

Reference is made to FIG. 3 exemplifying a system 100 configured as a sequential and/or continuous enantioselective reactor for selective production according to some embodiments of the invention. The system utilizes a magnetized structure 50 formed by one or more ferromagnetic or paramagnetic substrates (marked as FM). The substrate 50 may be coated with a thin capping or anchoring layer of diamagnetic material (e.g. gold). The magnetized substrate 50 is mounted on a moving platform 60, exemplified herein as including Belt 62 moved by Motor 64, configured to selectively transfer the substrate between several containers e.g. containers 72, 74 and 76. The containers carries solutions including selected reactants or solution selected for removing reaction products from the substrate 50.

As exemplified in FIG. 3, the magnetized substrate 50 structure is transferred and dipped in a container 72 (e.g. beaker) containing molecules of type A (e.g. Thiolate molecules A or TM(A)) selected as having suitable binding group that can bind to the magnetized substrate 50 surface. As a result of this process, a self-assembled monolayer of A molecules is formed on the magnetized substrate. The magnetized substrate 50 coated with the adsorbed molecules is then moved into a second container 74 holding a racemic mixture of chiral molecules B (e.g. racemic mixture containing enantiomers at certain ratio) that react with molecules A. Magnetization of the magnetized structure 50 provides enantio-specific reaction where only molecules B of one handedness interact with molecules A, forming an A-B product, while the others remain in the mixture. Generally, the substrate 50 may be placed in this container 74 for a selected time (e.g. 2-5 minutes) to limit interactions of the less preferred enantiomers.

To remove the reaction products from the magnetized substrate 50, it may be transferred to a third container 76 including solvent material. The A-B product molecules are removed from the magnetized structure 50 and can be moved for further processing or use. At this stage, the A-B reaction products generally contain a high ratio of one enantiomer (e.g. concentration ratio of 0.8 to 1 of a selected enantiomer). The process may be repeated for production of further complex molecules and it may include additional stages such as washing the magnetized structure. It should also be noted that the magnetized structure may be used as electrode for one or more electrochemical reactions with reactants of selected handedness. This is exemplified in FIG. 3 by container 76 that includes additional electrode 80 indicating selected electric potential differences (e.g. 0.9V) with the substrate. In such configurations, an electric connection is provided to the magnetized structure 50 and used for providing selected electric potential at the time the magnetized structure is located within one or more selected container for electrochemical reaction. Further, it should be noted that this reaction system configuration is not limited to covalent interaction and may be used for covalent reaction, or non-covalent interaction, as well as electrochemical interaction and “click chemistry” reactions.

The system 100 exemplified in FIG. 3 may also be used for continuous separation of chiral molecules from racemic mixture to provide enantiomer pure solutions. To this end the magnetized substrate is placed in a container 74 including the racemic mixture for a selected time (e.g. two minutes) to allow adsorption of molecules, while promoting adsorption of molecules of selected enantiomer. The substrate is then removed from the racemic container 74 and moved to a second container 76 to remove the adsorbed molecules, e.g. by washing or allowing the molecules to be released. This process removed a portion of molecules of certain handedness of the racemic mixture and transfer these molecules to another container. As the process is repeated, e.g. by transferring the same substrate or using plurality of substrates one by the other, the molecules are separated to enantiomers of one handedness in the initial container and those of the other handedness in the second container.

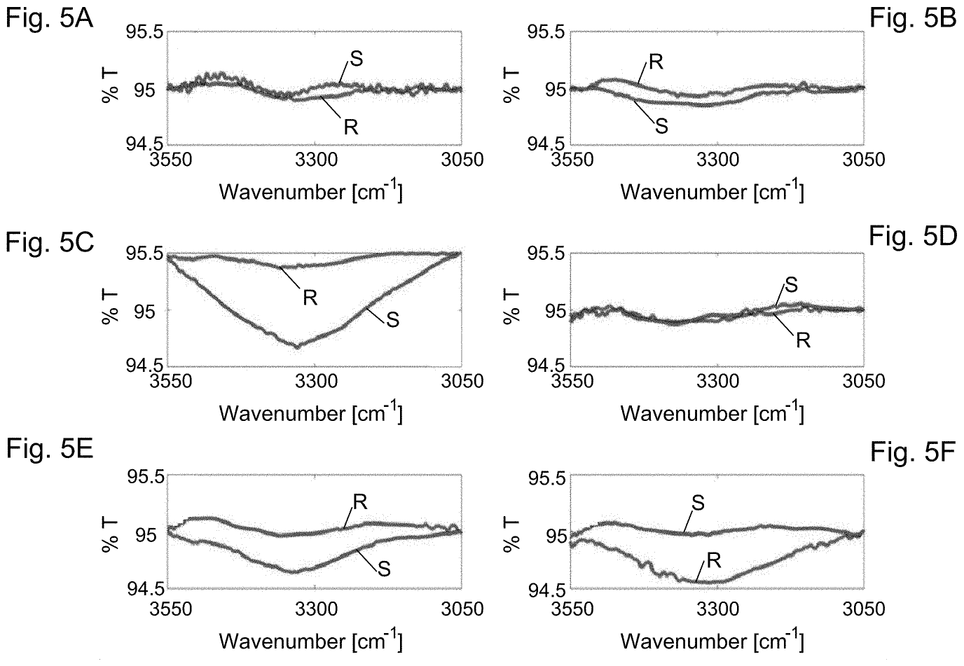

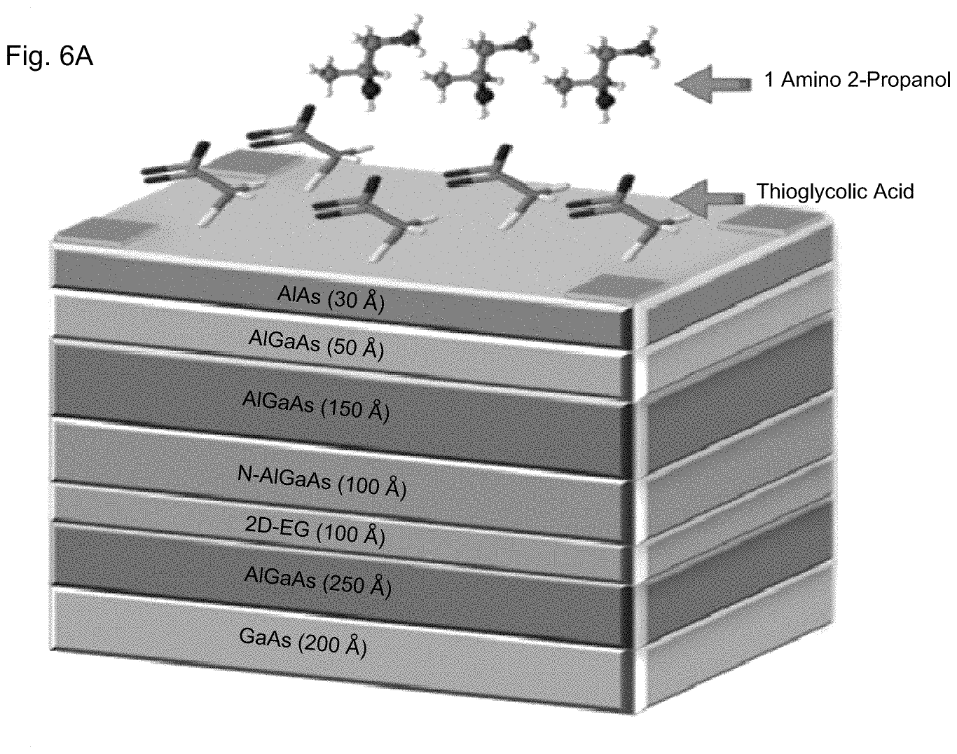

Reference is made to FIGS. 4A to 4B and FIGS. 5A-5F showing an experimental setup (FIG. 4A) and Fourier Transform Infrared spectroscopy (FTIR) results (FIG. 4B and FIGS. 5A-5F) illustrating the present technique. The system illustrated in FIG. 4A is designed for determining relation between interaction distance and enantioselectivity provided by the present technique. In this connection, the system utilizes a magnetized substrate (e.g. paramagnetic or ferromagnetic substrate marked in FIG. 4A as magnetized FM substrate) on which chiral molecules are linked (or adsorbed) forming a passivation monolayer of linker molecules of selected length. In this specific and non-limiting example, the magnetized substrate is made of 5 nm ferromagnetic nickel layer coated with 10 nm gold and may include additional layers as exemplified in FIG. 4A. The organic passivation layer operates to link the chiral molecules to the magnetized substrate, generally via symmetric linkers. The chiral molecules used in this example were (R) and (S) 1-amino-2-propanol. Further, in this specific and non-limiting example, the organic passivated monolayer was prepared as follows: 4×4 mm of ferromagnetic samples (n-type Si wafer, etched with 10% HF, and then coated with 12 nm Cr, 100 nm Au, 50 nm Ni and nm Au) were cleaned by boiling in ethanol for 10 min, then immersed in 10 mM monolayer molecule (e.g. thioglycolic acid, mercaptopropionic acid, mercaptooctanoic acid, mercaptobutiric acid, mercaptohexanoic acid and mercaptooctanoic acid) in EtOH for at least 2 hours. The monolayer adsorbed wafers were reacted with R or S enantiomers of 1-amino-2-propanol under magnetic field of 380 gauss in direction up or down with respect to surface (Au surface) of the substrate.

The distance between the magnetized ferromagnetic substrate and the chiral molecules was achieved and controlled by selecting the length of the linker molecules. More specifically, the linker molecules, forming a monolayer of stationary phase, were composed of molecules with the general structure HS—(CH₂)_{X-1}—COO⁻, where the thiol group acts to link the molecules to the substrate and the number X of carbon groups determined length of the linker. The tests were conducted several times using linker molecules of different lengths, i.e. different numbers X of carbon groups. The easy axis in this ferromagnetic Ni layer is set to be perpendicular to the substrate and it can be magnetized at the up direction (+) or down direction (−). The interaction of the chiral molecules with the stationary monolayer is based on electrostatic bonds. Two enantiomers of R or S 1-amino-2-propanol were prepared in ethanol (pH˜7) solvent for all or most experiments. In this situation of about pH-7, both the carboxyl group and the amine group are charged, so that an electrostatic interaction between them is possible. The hydroxyl group, apart from defining the chiral center, acts as an Fourier-transform infrared spectroscopy (FTIR) marker for stretch modes in the wavenumber range of 3200-3500 cm⁻¹.

FIG. 4B shows FTIR results after a reaction time of two-minutes under different directions of magnetization of the nickel layer. The FTIR measurements are collected from a clean gold surface (clean) without the use of linker molecules, a surface with a thioglycolic acid (TGA), and two sample surfaces with S 1-amino-2-propanol where the reaction was performed with opposite magnetization directions (with the magnetic pole is pointing up for “plus Dir”, or down for “minus Dir”). Each experiment was repeated minimal 3 times to ensure consistency. The OH stretch signal appears in S enantiomer under down (minus Dir) magnetization direction at about 3300-3450 cm⁻¹ and stretching the FTIR graph to lower % T values, indicates interaction with the linker molecules. FIGS. 5A to 5F show additional FTIR results collected from the sample surfaces with S 1-amino-2-propanol, marked (S) and R 1-amino-2-propanol, marked (R) for different reaction times with up (+) or down (−) magnetization directions. More specifically, FIG. 5A shows FTIR results after 30 seconds interaction time with magnetic field directing down; FIG. 5B shows FTIR results after 30 seconds interaction time with magnetic field directing up; FIG. 5C shows FTIR results after 2 minutes interaction time with magnetic field directing down; FIG. 5D shows FTIR results after 2 minutes interaction time with magnetic field directing up; FIG. 5E shows FTIR results after 5 minutes interaction time with magnetic field directing down; and FIG. 5F shows FTIR results after 10 minutes interaction time with magnetic field directing up.

The OH stretch signals appears in S enantiomer under down (−) magnetization direction at about 3300-3450 cm⁻¹ after 2-minute interaction in FIG. 5C stretching the FTIR graph to lower % T values and remains after 5-minute interaction in FIG. 5E indicates interaction with the linker molecules. For the R enantiomer, the OH stretch appears at similar wavenumbers for up (+) magnetization after 10-minute interaction in FIG. 5F. Thus, the selective adsorption of S enantiomer is generated faster compared to R enantiomer for corresponding magnetization directions. More specifically, after 2 minutes molecules of the S enantiomer are selectively absorbed, while molecules of the R enantiomer show selective adoption after 10 minutes interaction, even when the magnetic field is of the corresponding direction. The hydroxyl (OH) stretch is visible within the expected range of 3300-3450 cm⁻¹. The spectra show that the chirality (R/S) of the analyte adsorbed is in agreement with the magnetization direction. These selective reactions are maintained for about 30-45 minutes where R enantiomer adsorbed mostly under up (+) magnetization direction and S enantiomer only under down (−) magnetization direction. After longer interaction time, the spin polarization generally equilibrates and both enantiomers may interact with the linker monolayer showing no difference in adsorption between the two enantiomers.

By changing the linker length, location of the interacting ends of the molecules with respect to the magnetized surface changes. Increased linker lengths results in decay of spin polarization of the electrons participating in the interaction, reducing selectivity of the interaction. The use of linker molecules of varying lengths enables to monitor this effect. The inventors have found that the decay of the selectivity is clearly noticed with linker length of more than 3 carbons (X=3), which relates to linker length of 6.3 Å under assumption of tetrahedral angle and 0.77 Å covalent radius of carbon in c-c bonding. Thus, the selectivity decays at distances larger than 6.3 Å. With any longer linkers, the interaction does not show selectivity with respect to chirality of the molecules and magnetization of the substrate. Thus, the adsorption rate, using linkers longer than 6.3 Å is similar for up or down magnetization and R or S enantiomers. At this case, the interaction shows similar characteristics to the case where no magnetization is applied on the surface. The system was verified also for mercaptohexanoic acid (with X=6) and mercaptooctanoic acid (with X=8). Both did not show any qualitative difference, there was no selective adsorption and the analyte adsorbed in the same rate under up or down or without magnetization. These results suggest that the length dependence decay is due to losing the electron spin coherency in the linker molecules biased by the magnetic field on the ferromagnet.

As indicated, at large distance (longer than length of 3 carbons' covalent bonds) electrostatic bonds may be created at the same rate with and without magnetic field for the two enantiomers. At these conditions the OH stretch signal appears after 30 minutes, indicating time of interaction between the 1-amino-2-propanol molecules and the linker molecules. Generally, if the interaction with the substrate is stronger, the first signal of the OH stretch is expected to appear faster, while if it is weaker the signal is expected to appear after longer interaction time. Table 1 exemplifies minimal absorption durations for different linker lengths and magnetizations, normalized by the time of adsorption with long linkers where enantioselectivity vanishes. More specifically, the time of the appearance of the OH stretch signal in FTIR measurements for interaction with short linkers is normalized by the time of appearance of the OH stretch signal after interaction with long linkers where the substrate magnetization does not play a role. In table 1, the first and the second rows are the normalized times relevant to reaction of R 1-amino-2-propanol, and the third and fourth last rows are the normalized times relevant to reaction of S 1-amino-2-propanol. The interaction rates were correlated to the first appearance of the OH stretch signal, defined by integrating the peak area having a signal that is two folds higher than that shown in FTIR of the TGA monomers. The measured time was measured and averaged over 5 samples. The time under up (+) magnetization direction for the R enantiomer is faster than under down (−) magnetization direction. The S enantiomer displays the opposite trend. When 4 carbons (7.5 Å) are used, no selective absorbance is achieved.

TABLE 1

| Analyte, condition/ | Thioglycolic | Mercaptopropionic | Mercaptobutiric |
|---|---|---|---|
| Stationary phase | acid | acid | acid |
| (monolayer) | (2 Carbons ~5 Å) | (3 Carbons ~6.3 Å) | (4 Carbons ~7.5 Å) |
| (R)-1-Amino-2- | ⅓ | ½ | 1 |
| propanol magnetic | | | |
| field direction (+) | | | |
| (R)-1-Amino-2- | 3 | 1.5 | 1 |
| propanol magnetic | | | |
| field direction (−) | | | |
| (S)-1-Amino-2- | 2 | 3 | 1 |
| propanol magnetic | | | |
| field direction (+) | | | |
| (S)-1-Amino-2- | 1/15 | ⅓ | 1 |
| propanol magnetic | | | |
| field direction (−) | | | |

As shown in table 1, the use of short linking layers to magnetized surface provide enantioselective interactions. As indicated above, this is considered to be associated with the CISS exchange interactions being stronger due to spin polarization of interacting electrons. For enantiomers of selected handedness, weaker interaction is shown as well as longer adsorption rates for one magnetization direction, while in the opposite magnetization direction, shorter adsorption rates are shown. The enantiomers of opposite handedness behave similarly for opposite magnetization directions. The shorter the linkers the selectivity becomes stronger. For linkers having length associated with 2 carbon bonds, the selectivity reaches a factor of 9 for the R enantiomer and a factor of 30 for the S enantiomer. For linker length of 3 carbons, the selectivity reaches a factor of 3 for the R enantiomer and a factor of 9 for the S enantiomer. With 4 carbons linkers, the selectivity disappears.

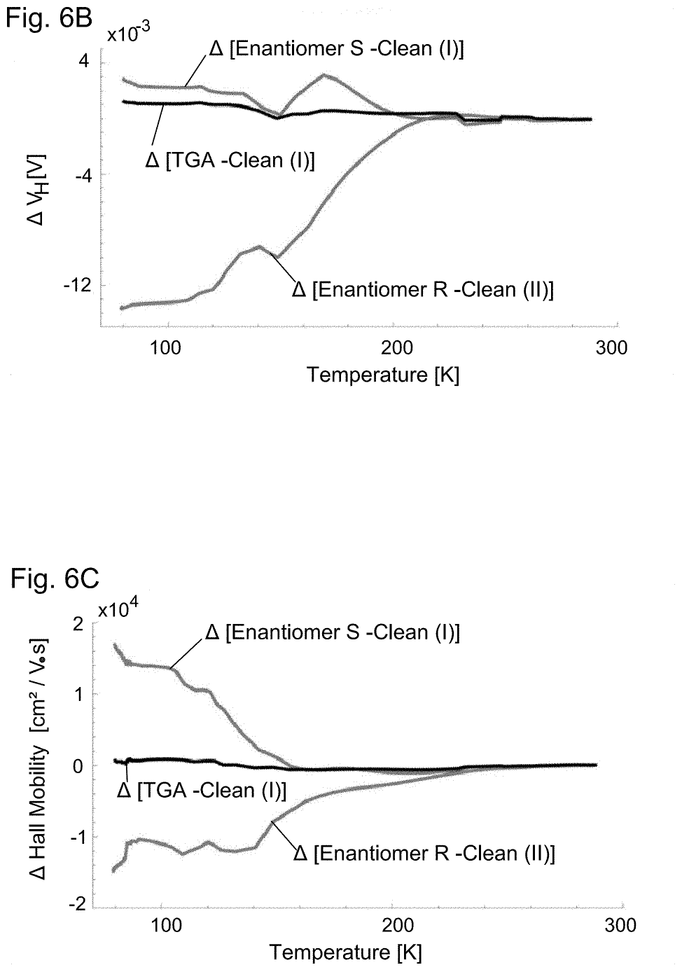

To relate the asymmetrical electrostatic adsorption properties to magnetic spin, the same chemical layout was deposited on sensitive Hall devices based on GaAs/AlGaAs heterostructure with shallow 2D electron gas. Reference is made to FIGS. 6A to 6C illustrating an example of the device configuration including a layered Hall device and Hall voltage measurements thereof. This device exemplified in FIG. 6A is configured to provide mobility improvements and high sensitivity measurements for magnetization effects occurring on the surface. The device configuration utilizes interaction of S/R 1-amino2-propanol with thioglycolic acid (TGA) on top of layered structure including 2D electron gas based on gallium arsenide heterostructure chip. In a specific and non-limiting example, van der Pauw devices were fabricated using lithography technique on a GaAs/AlGaAs layers substrate with 2DEG (two-dimensional electron gas) layer embedded providing high electron mobility. Alloy of Ni/Au/Ge/Ni/Au was evaporated on the structure forming 4 corner contact pads, followed by liftoff in n-methyl-2-pyrrolidone (NMP) and later annealing at 450° C. in order to diffuse the contacts into the 2DEG. Next, the device was etched with citric acid to remove the upper conduction layer and finally ohmic contacts were confirmed by standard resistance measurement. A monolayer of TGA was first adsorbed in EtOH immersing the device in 10 mM solution. Selected chiral molecules R/S 1-amino2-propanol were dissolved in ethanol (1 mM solution) and adsorbed on top of the TGA linker layer.

FIG. 6B shows the difference in Hall voltage values as measured on a clean device with no adsorbed 1-amino-2-propanol (i.e. only TGA), and after the absorption of chiral molecules (enantiomer S & enantiomer R). At each step a set of Hall measurements was performed applying magnetic field of 0.8 T and 30 μA current. An initial temperature of about 80 K was set using liquid N₂ and the temperature was measured periodically until the temperature raise to 290 K. The chiral molecules were removed from one of the devices by a quick wash using water and ethanol and then chiral molecules of the opposite orientation (handedness) were adsorbed. The latter adsorption was done in the same technique as the former one and once again Hall measurements were conducted. The curve S refers to the S-enantiomer and the curve R refers to the R-enantiomer. In this test, the TGA-clean sample presents a reference describing the difference in measurement before and after adsorption of thioglycolic acid. FIG. 6C shows the same presentation for Hall mobility in additional measurements. The measurements of FIGS. 6B and 6C show that the R/S enantiomers adsorption induce opposite Hall voltage (FIG. 6B) as well as opposite Hall mobility (FIG. 6C) between the different enantiomers. Since the bonds are electrostatic, the effect is reversible. The device was first synthesized and measured with the linkers layer for background subtraction. Then, the S enantiomer was added, and the device was measured again. Lastly, after gentle washing of the S enantiomer, the R enantiomer was attached. With one enantiomer orientation, an increase in Hall voltages was measured, while the other orientation shows a decrease in Hall voltages. The Hall effect results strengthens that CISS effect and spin exchange interaction controls the adsorption selectivity. This is also true for interaction through short linkers and physical electrostatic bonds.

The results above indicate stereo-chemistry and enantioselective reactions provided by promoting physical and chemical molecular interactions at the vicinity of magnetized surfaces. More specifically, the present technique provides enantioselective interactions on, or at selected distances (generally not exceeding 0.7 nm) from magnetized surface (being ferromagnetic or paramagnetic surface). Furthermore, the enantio-selective interactions are stable for relatively long period of time, in response to adsorption onto the magnetized surface.

Thus, the present technique provides for promoting enantioselective interaction between selected chiral molecules. Generally, unlike the conventional techniques using chromatographic systems with selected lock and key features that are specific for selected molecules. The present technique enables the use of physical bonds and CISS controlled reaction that provide generic differentiation between enantiomers, where similar system configuration provides enantioselective interactions to various reactants. Further, the present technique is also relevant for selective interactions using chiral, helical as well as non-chiral and non-helical molecules, where in the later cases, the selectivity is manifested in chirality of the reaction products. Accordingly, the present technique demonstrates spin-based chemistry, exemplifying selected enantioselective interactions where the interaction centers are on, or in vicinity of a surface having magnetization perpendicular to the surface.

1. A system for use in synthesis of molecules, comprising: a container configured for containing fluid mixture comprising one or more reactant molecules, and at least one surface comprising ferromagnetic or paramagnetic material, located to be in at least partial contact with reactants in said container, said ferromagnetic or paramagnetic material being magnetizable with magnetization direction perpendicular to said at least one surface, thereby providing chiral selective synthesis from said one or more reactant molecules.

2. The system of claim 1, wherein said at least one surface comprises structured substrate comprising at least one layer of ferromagnetic or paramagnetic material.

3. The system of claim 2, wherein said structured substrate comprises at least one surface layer having affinity for adsorption with one or more selected reactants, thereby enabling molecular interactions between reactants at vicinity of the at least one surface.

4. The system of claim 3, further comprising at least one of: (i) at least one type of reactant molecules pre-adsorbed on said at least one surface, the pre-adsorbed reactant molecules being capable of selectively interacting with molecules of selected handedness in said fluid mixture or (ii) one or more additional types of reactant molecules being absorbed in corresponding one or more additional layers associated with enantiospecific interactions.

5. (canceled)

6. The system of claim 1, wherein said at least one surface is associated with an electrode providing electrical contact with said fluid mixture, thereby promoting electro-chemical reaction at vicinity of the at least one surface.

7. The system of claim 1, wherein said at least one surface comprising ferromagnetic or paramagnetic material is mounted to be selectively placed to be in contact with reactants in said container, and to be placed out of contact with said reactants.

8. The system of claim 1, configured for selectively promoting chemical reactions for enantiomers of selected handedness of chiral molecules in accordance with direction of magnetization perpendicular to said at least one surface being up or down with respect to said at least one surface.

9. A system for use in synthesis of molecules comprising:
a container configured for containing fluid mixture that comprising one or more types of chiral molecules;
an electrode arrangement comprising at least first and second electrodes, being configured for applying an electric field on said fluid mixture; said first electrode comprises at least one ferromagnetic or paramagnetic material providing at least one interface with said fluid mixture, said at least one ferromagnetic or paramagnetic material being selectively magnetized in up or down direction perpendicular to surface of said first electrode; said second electrode being located at a predetermined distance from said first electrode and being electrically insulated therefrom; wherein magnetization of said first electrode provides spin selectivity in electrons transmission in interaction with molecules in said fluid mixture, thereby providing enantioselective interaction with molecules in said fluid mixture; and
a power unit configured for providing electrical voltage and current between said first and second electrodes.

10. The system of claim 9, wherein said first electrode comprises at least one ferromagnetic or paramagnetic material, said system further comprises at least one field generating unit configured to selectively apply magnetic field onto said first electrode providing selected magnetization direction of said first electrode.

11. The system of claim 9, configured for at least one of synthesis or separation of chiral molecules or at least one enantiospecific reaction such of selected enantiomer from racemic mixture.

12. (canceled)

13. The system of claim 11, wherein the enantiospecific reaction is electrochemical oxidation or reduction reactions.

14. The system of claim 9, wherein at least said first electrode is mounted to be selectively insert into said container for selectively start and stop said interaction with reactants in said fluid mixture.

15. A method for use in molecular synthesis, the method comprising: providing at least one substrate having magnetization perpendicular to surface of said substrate; bringing said substrate in contact with one or more types of reactant molecules for promoting one or more molecular reactions on or in vicinity of surface of said substrate; wherein magnetization direction of said at least one substrate provides spin selectivity of electrons participating in said one or more molecular reactions resulting in enantioselective reactions.

16. The method of claim 15, wherein said one or more molecular reactions comprise electro-chemical reaction, the method further comprises applying selected electrical voltage on said at least one substrate thereby receiving or donating electrons of selected spin polarization to perform said one or more molecular reactions.

17. The method of claim 15, further comprising adsorbing one or more selected reactants onto said surface of said at least one substrate prior to bringing said substrate in contact with one or more types of reactant molecules, thereby providing enantioselective interactions between said one or more types of reactant molecules and said one or more selected reactants adsorbed on said surface.

18. The method of claim 15, wherein said one or more types of reactant molecules comprise chiral molecules, said enantioselective reactions provide at least one of reactions of enantiomers of one handedness selected in accordance with direction of magnetization of said at least one substrate or formation of chiral products having handedness selected in accordance with direction of magnetization of said at least one substrate.

19. (canceled)

20. (canceled)

21. The method of claim 18, further comprising transferring said at least one substrate to container comprising buffer solution for removing molecules of said selected handedness from said at least one substrate to enable enantiomer separation by physical adsorption enantiomers of selected handedness.

22. A method for use in separation of chiral molecules, the method comprising: providing at least one substrate having magnetization perpendicular to surface of said substrate; bringing said substrate in contact with mixture of chiral molecules for a selected time and removing said substrate from said mixture to remove the adsorbed molecules therefrom; wherein magnetization direction of said at least one substrate provides preferred adsorption of molecules of one enantiomer over molecules of the opposite enantiomer thereby removing molecules of a selected enantiomer from said mixture.

23. The method of claim 22, further comprising at least one of washing adsorbed molecules from said substrate in a second container, thereby providing a solution of substantially enantiomer pure mixture or repeatedly bring said substrate into contact with said mixture of chiral molecules for selected time and washing said substrate in a second container to thereby provide a continuous separation process.

24. (canceled)

25. The method of claim 22, comprising using a plurality of substrates and repeating said separation process with each of said substrates.
